(19) United States
(12) Patent Application Publication
(10) Pub. No.: US 2024/0358994 A1
(43) Pub. Date: Oct. 31, 2024

(54) IN-VIVO MICRO BLOOD PUMP

(71) Applicant: INVENTURE TECH LTD, Oxford (GB)

(72) Inventors: Daniel ZAJARIAS FAINSOD, Haifa (IL); Ilan MARCUSCHAMER, Ramat Gan (IL)

(73) Assignee: INVENTURE TECH LTD, Oxford (GB)

(21) Appl. No.: 18/566,055

(22) PCT Filed: Jun. 1, 2022
(86) PCT No.: PCT/IL2022/050583

§ 371 Date: Nov. 30, 2023

Related U.S. Application Data
(60) Provisional application No. 63/195,763, filed on Jun. 2, 2021.

(51) Int. Cl. A61M 60/135; A61M 60/216; A61M 60/422; A61M 60/546

(52) U.S. Cl. CPC A61M 60/135; A61M 60/216; A61M 60/422; A61M 60/546; A61M 2205/3331

(57) ABSTRACT

An in-vivo micro blood pump is disclosed. The pump, comprising: a holder configured to be inserted into a blood vessel to be anchored to the blood vessel by one or more stents; and two or more micro-impellers located inside the holder while having the same rotation axis. Each micro-impeller may include a rotor and a cylindrical stator. The rotor may include a first hollow cylinder; two or more internal blades, extending inward from a wall of the first hollow cylinder, wherein the radial dimension of each blade is less than an internal radius of the first hollow cylinder; and one or more magnets. The cylindrical stator may include one or more electromagnets. A first micro impeller has a first blade pitch, and a second micro impeller has a second blade pitch, different from the first blade pitch.

### CROSS-REFERENCE TO RELATED APPLICATION

This application claims the benefit of priority of U.S. Provisional Patent Application No. 63/195,763, titled “IN-VIVO MICRO BLOOD PUMP”, filed Jun. 2, 2021, the contents of which are incorporated herein by reference in their entirety.

### FIELD OF THE INVENTION

The present invention relates generally to blood pumps. More specifically, the present invention relates to in-vivo micro blood pumps.

### BACKGROUND OF THE INVENTION

Micro blood pumps are miniature flow assisting devices configured to be inserted into an artery, during catheterization, for example, when treating an advanced heart failure (AdHF) patient. Most micro blood pumps include a rotor, blades, diffuser, and stator. The various pumps vary in several parameters, each being designed to solve specific problems in AdHF, for example, rotor diameter, number of blades, inlet and outlet angles, blade height, volute or diffuser diameter, and operating speed.

However, all known micro blood pumps are single devices designed to cause a single blood flow rate, with no flexibility. Currently, available solutions are single-stage impeller systems; the impeller is either large, usually centrifugal, and requires extra cardiac implantation or are smaller axial impellers that may be implanted intracardiac or intravascularly. The former are able to run at lower rotational speeds and therefore lower sheering forces and consequently produce less hemolysis or rupture and activation of blood cells reducing the incidence of anemia, blood clotting and consequently pump failure: these devices are used as a bridge or alternative to heart transplantation as they are tolerated for longer periods of time. Conversely, the latter may be easily implanted without the need for open-heart surgery, however, to achieve high flow outputs requires running at very high rotational speeds and therefore produce hemolysis and clotting leading to severe anemia and clot formation and hence may only be used for short periods of time of up to several days, for example as a method of assisting the heart muscle recovery after damage by heart disease or surgical intervention. Moreover, these aforementioned pumps have a fixed rotational speed and flow and do not adapt the output according to the requirements of the patient during different levels of physical activity.

Accordingly, there is a need for more flexible, minimally invasive, and long-term pumps using a multi-stage impeller system, that can produce variable flowrates according to the patient's physical requirements whilst reducing the deleterious effects caused by high-speed blades.

### SUMMARY OF THE INVENTION

Some aspects of the invention may be directed to an in-vivo micro blood pump, comprising: a holder, configured to be inserted into a blood vessel to be anchored to the blood vessel by one or more stents; and two or more micro-impellers located inside the holder while having the same rotation axis. In some embodiments, each micro-impeller may include a rotor and a cylindrical stator. In some embodiments, the rotor may include a first hollow cylinder; two or more internal blades, extending inward from a wall of the first hollow cylinder, wherein the radial dimension of each blade is less than an internal radius of the first hollow cylinder; and one or more magnets. In some embodiments, the cylindrical stator may include one or more electromagnets. In some embodiments, a first micro impeller has a first blade pitch, and a second micro impeller has a second blade pitch, different from the first blade pitch.

In some embodiments, the one or more magnets are embedded in the walls of the first hollow cylinder. In some embodiments, the one or more electromagnets are embedded in a second hollow cylinder included in the cylindrical stator, such that, when the electromagnets are provided with electrical power the rotor magnetically levitates.

In some embodiments, the holder is one of: a tube, a mesh and a stent.

In some embodiments, the in-vivo micro blood pump may further include a driveline configured to provide communication signals and electrical power to the two or more micro-impellers. In some embodiments, the in-vivo micro blood pump may further include a controller configured to control the rotating speed of all the micro impellers. In some embodiments, the in-vivo micro blood pump may further include one or more pressure sensors; and a controller configured to control the rotating speed of at least one rotor based on a signal received from the one or more pressure sensors.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A, 1B, 1C and 1D are illustrations of in-vivo micro blood pumps according to some embodiments of the invention;

FIGS. 2A, 2B, and 2C are illustrations of impellers according to some embodiments of the invention;

FIGS. 3A, 3B, 3C, and 3D are illustrations of in-vivo micro blood pumps, before and after anchoring, according to some embodiments of the invention;

FIGS. 4A and 4B are illustrations of two stages in the implantation procedure of an in-vivo micro blood pump according to some embodiments of the invention; and

FIGS. 5A, 5B, 5C, 5D, 5E, and 5F are illustrations of stages in the implantation procedure of an in-vivo micro blood pump according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Aspects of the invention may be directed to an in-vivo micro blood pump. Such an in-vivo micro blood pump may include multiple stages, each stage may include a different impeller configured to provide a different flow rate.

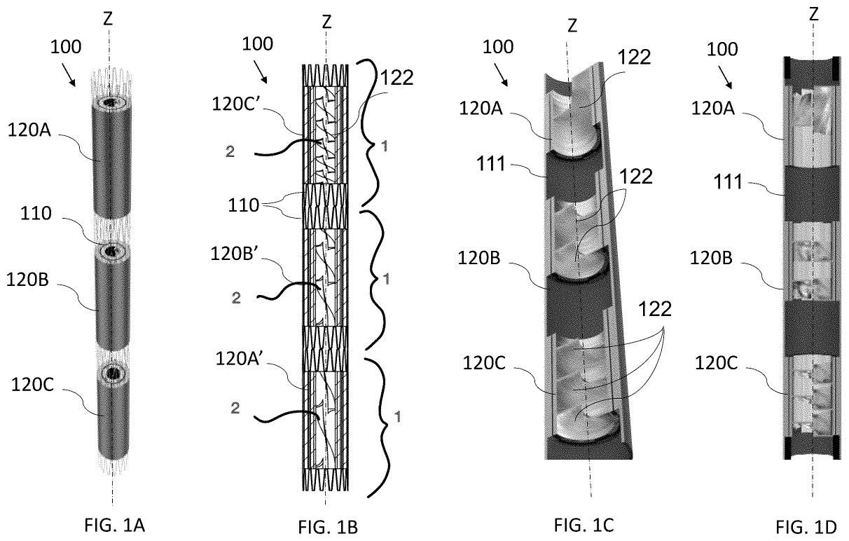

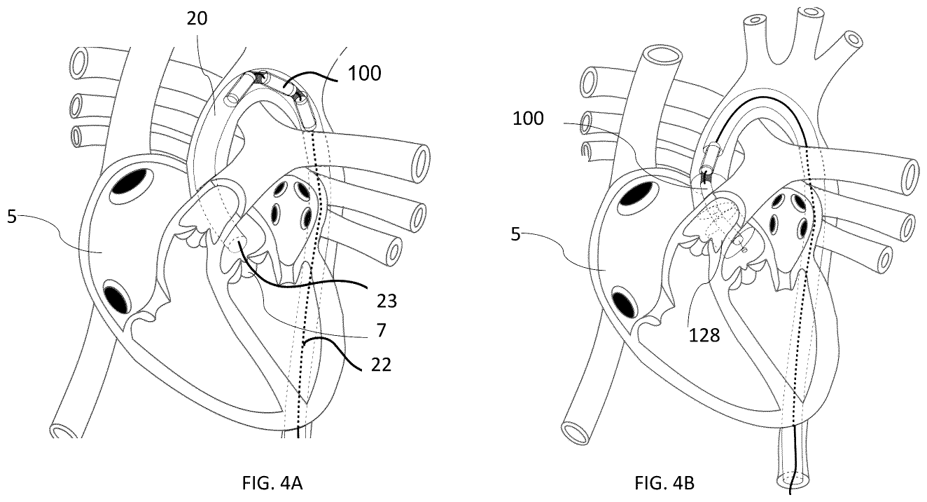

Reference is now made to FIGS. 1A, 1B, 1C, and 1D which are illustrations of in-vivo micro blood pumps according to embodiments of the invention. FIG. 1A is an illustration of a three-dimensional (3D) model of an in-vivo micro blood pump according to embodiments of the invention. FIG. 1B is a drawing of a cross-section view of an in-vivo micro blood pump according to embodiments of the invention and FIGS. 1C and 1D are cross-sections in a 3D model of an in-vivo micro blood pump according to embodiments of the invention. An in-vivo micro blood pump 100 may include a holder 110 or 111, configured to be inserted into a blood vessel and to be anchored to the blood vessel by one or more stents or struts. A process of inserting, anchoring, and implanting in-vivo micro blood pump 100 in a blood vessel, in this particular illustration an artery, is discussed with respect to FIGS. 4A and 4B. Holder 110 may include a tubular mesh configured to hold micro impellers 120A, 120B, 120C, 120A′, 120B′, and 120C′. In some embodiments, holder 110 may include at least one stent configured to anchor pump 100 into the blood vessel. In some embodiments, holder 111 may be a full tube configured to hold one or more micro-impellers 120A, 120B, 120C.

In some embodiments, two or more (e.g., three as illustrated) micro-impellers 120A, 120B, 120C, 120A′, 120B′, and 120C′ located inside holder may have the same rotation axis Z. In some embodiments, axis Z may not necessarily be a straight line, and can be the central line of a catheter holding pump 100 inside a blood vessel, as illustrated in FIGS. 4A and 4B. In some embodiments, a first micro impeller 120A may have blades with a first pitch, and a second micro impeller 120 B may have blades with a second pitch, different from the first pitch. For example, micro impeller 120A may have a larger pitch than micro impeller 120B which in turn may have a larger pitch than micro impeller 120C. In the nonlimiting example illustrated in FIGS. 1A-1D the size of the pitch in micro impellers may be determined by the number of blades 122 since all the micro impellers have the same length. For example, micro impeller 120A may have a single blade, micro impeller 120B may have two intertwining blades (e.g., a double helix) and micro impeller 120C may have three intertwining blades (e.g., a triple helix). In another nonlimiting example, of FIG. 1, micro impeller 120A′ may have a single blade, micro impeller 120B′ may have three intertwining blades and micro impeller 120C′ may have five intertwining blades. Additionally, the pitch between the turns of the blades may also vary between the different impellers.

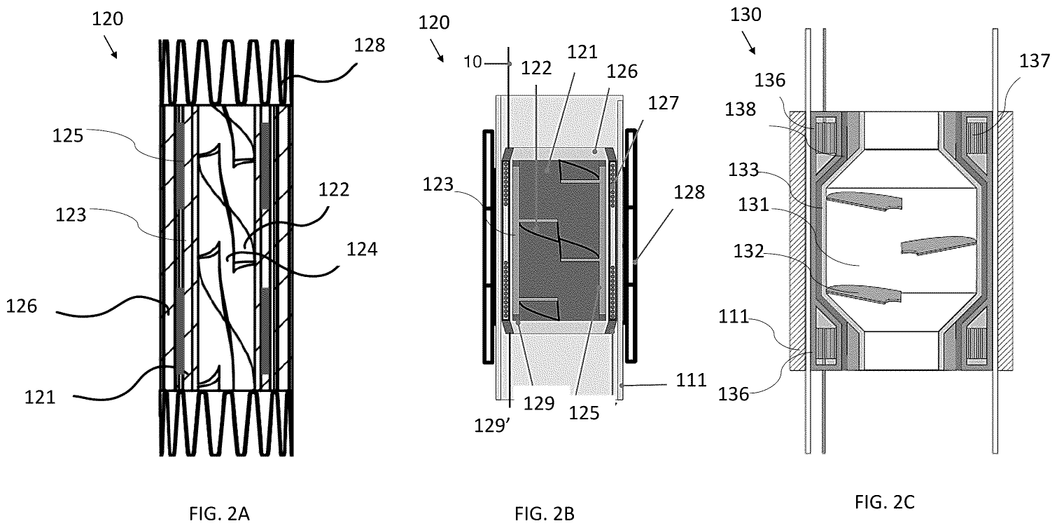

Reference is now made to FIGS. 2A and 2B which are detailed illustrations of impellers according to some embodiments of the invention. A micro impeller 120 may include any number of blades and may have any pitch and may be configured to be assembled in in-vivo micro blood pump 100. Micro-impellers 120A, 120B, 120C, 120A′, 120B′, and 120C′ may have substantially the same components as micro impeller 120 and may differ from each other in the pitch and/or number of blades.

Micro impeller 120 illustrated in FIGS. 2A and 2B may include a rotor 121 comprising a hollow cylinder 123 and one or more internal blades 122, extending inward from a wall of hollow cylinder 123, such that the radial dimension of each blade is less than an internal radius of hollow cylinder 123. Such an arrangement may form a central space 124 inside the impeller along its entire length. In some embodiments, rotor 121 may further include one or more magnets (e.g., static magnets) 125. In some embodiments, one or more magnets 125 may be embedded in the walls of hollow cylinder 123, for example, using an additive manufacturing process. In some embodiments, rotor 121 may further include a terminal magnetic bearing 129.

Micro impeller 120 may further include a cylindrical stator 126 that may include electromagnets 127 (e.g., electromagnetic coils). In some embodiments, stator 126 may further include a terminal magnetic bearing 129′. In some embodiments, when electromagnets 127 are provided with electrical power rotor 121 may magnetically levitate and rotate, for example, inside cylindrical stator 126. In some embodiments, the one or more electromagnets are embedded in a second hollow cylinder included in the cylindrical stator.

In some embodiments, rotor 121 and hollow cylinder 123 as well as cylindrical stator 126 may be composed of a biocompatible material or metal alloy, with or without a biocompatible coating. It may be manufactured by a combination of different methods including additive manufacturing such as selective laser sintering or conventional manufacturing methods. In some embodiments, permanent magnets 125 contained in rotor 121 as well as forming the magnetic bearing 129 may be either manufacture by known conventional methods and inserted into rotor 121 and stator 126 or be incorporated to body 121 during additive manufacture by either placement or construction by a similar deposition of magnetized material in the additive manufacturing process.

In some embodiments, components including electromagnets 127 (e.g., coils) required in the stator component of the motor may also be incorporated to stator 127 during additive manufacturing or as a printed circuit board or produced separately by conventional manufacturing methods and inserted into the body of the stator. In some embodiments, one or more of the micro impellers 120 may further include a stent 128 for anchoring pump 100 (illustrated in FIGS. 1A-1D) into a blood vessel. For example, stent 128 may be made from shape-memory alloy such as NiTinol with or without a polytetrafluoroethylene or other biocompatible coating that expands to hold the impeller units in place inside the blood vessel (e.g., aorta or pulmonary artery, the vena cava and the like).

In some embodiments, anchoring stent 128, struts, or otherwise may be produced by weaving of a shape-memory alloy or other malleable material or by means of laser cutting which is then incorporated to the body of the stator by welding or other method of attachment.

In some embodiments, two or more micro impellers 120 may be connected by a tubular structure 111, which may or may not be porous or fenestrated, permitting two or more impellers to produce a progressive pressure head.

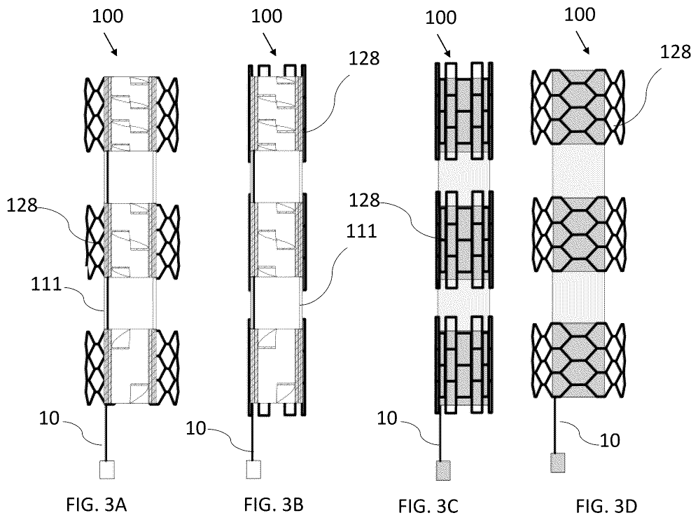

In some embodiments, in-vivo micro blood pump 100 (illustrated in FIGS. 3A, 3B, 3C and 3D) may include a driveline 10 configured to provide communication signals and electrical power to the motors of the micro-impellers 120.

Reference is now made to FIG. 2C which is an illustration of another impeller according to some embodiments of the invention. A micro impeller 130 may include any number of blades and may have any pitch and may be configured to be assembled in in-vivo micro blood pump 100. Micro-impellers 120A, 120B, 120C, 120A′, 120B′ and 120C′ may have substantially the same components as micro impeller 130 and may differ from each other in pitch and/or number of blades.

Micro impeller 130 may include a rotor 131 and a stator 136 concentric and exterior to rotor 131, such that the magnetic drive components may be repositioned near both ends of impeller 130 rather than along it to reduce the overall diameter of the impeller. Rotor 131 may include a hollow cylinder 133, permanent magnets 138 and one or more internal blades 132, extending inward from a wall of hollow cylinder 131, such that the radial dimension of each blade is less than an internal radius of hollow cylinder 133. Stator 136 may include electromagnets 137. In some embodiments, when electromagnets 137 are provided with electrical power, rotor 131 may magnetically levitate and rotate in stator 136.

Reference is now made to FIGS. 3A, 3B, 3C and 3D which are illustrations of in-vivo micro blood pumps 100, before and after anchoring, according to some embodiments of the invention. FIGS. 3A and 3B show a cross section view of in-vivo micro blood pumps 100 of FIGS. 3C and 3D. In FIGS. 3B and 3C stents 128 are folded closely to holder 111. In FIGS. 3A and 3D stents 128 are open, thus may anchor micro pumps 100 to the walls of a blood vessel. In an alternative embodiment the stents or alternative holder mechanism 128 (e.g. struts) May be placed concentric to communicating tube 111 as well as at both ends of both the first and last impeller unit. FIGS. 3A, 3B, 3C and 3D further illustrate driveline 10 configured to provide communication signals and electrical power to two or more micro-impellers included in pump 100.

In some embodiments, in-vivo micro blood pump 100 may further include a controller (not illustrated) configured to control the rotating speed of all the micro impellers, for example, by controlling the electrical power, frequency, or other parameter, provided to electromagnets 127. In some embodiments, in-vivo micro blood pump 100 may further include or may be in communication with one or more pressure sensors (not illustrated) and the controller may be configured to control the rotating speed of at least one rotor based on a signal received from the one or more pressure sensors. In some embodiments, the pressure sensors may be located in, for example, the right atrium (measuring preload), left atrium, left ventricle, and aortic root when placed in the aorta, or right atrium, right ventricle and pulmonary trunk when placed in the pulmonary artery. In another embodiment of the invention, micro-impeller pumps may be placed in multiple locations such as the pulmonary trunk and aortic artery providing biventricular support.

Reference is now made to FIGS. 4A and 4B which illustrate two stages in the implantation procedure of an in-vivo micro blood pump according to some embodiments of the invention. A nonlimiting example of the implementation procedure may include obtaining arterial access by, for example, either femoral artery by means of a Seldinger technique or similar, for placement in the aorta, or the femoral vein for placement in the pulmonary trunk. Moreover, techniques such as a temporary aorto-caval anastomosis for accessing the aorta by means of a central venous access route or alternative direct apical puncture do deliver the device in an anterograde manner from the ventricle to the aorta May be used. In some embodiments, fluoroscopic guidance may be used, for example, for guiding an 8 Fr catheter 20 (or appropriate size) to advance toward a heart 5 just distal to the aortic valve 7, using any known guidewire-catheter technique. Using any appropriate exchange technique, in-vivo micro pump 100 may be attached to a delivery wire 22, to be advanced inside catheter 20 using fluoroscopic guidance, as illustrated in FIG. 4A. In some embodiments in-vivo micro pump 100 may be advanced to the tip of the catheter sheath 23, as illustrated in FIG. 4B. In some embodiments, in-vivo micro pump 100 may be anchored using stents 128. In some embodiments, the delivery wire 22 may be detached from micro pump 100, for example, using Joule heating, or alternate method, and withdrawn from the body. Followed by the withdrawal of catheter 20.

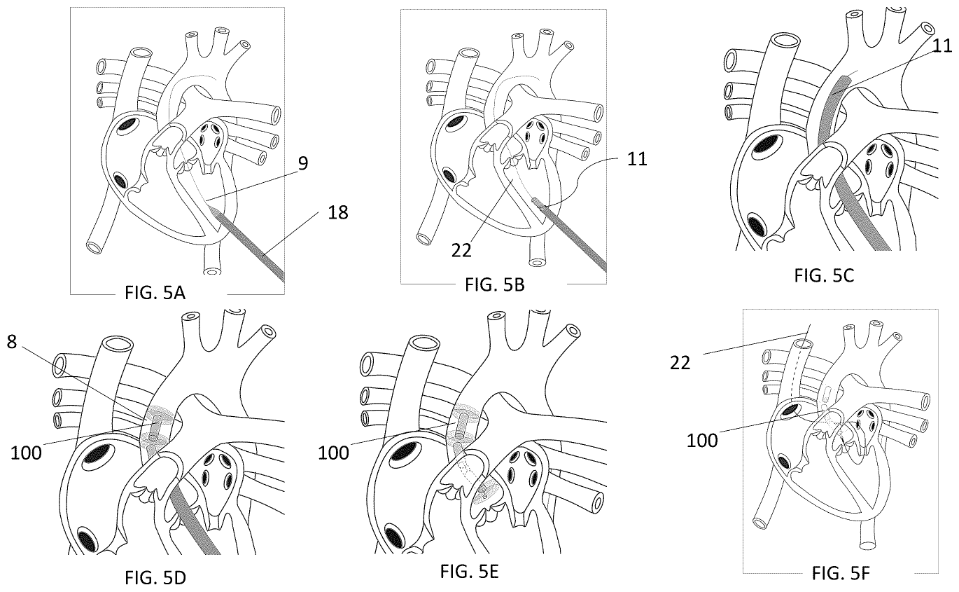

Reference is now made to FIGS. 5A, 5B, 5C, 5E and 5F which illustrates stages in another implantation procedure of an in-vivo micro blood pump according to some embodiments of the invention. In some embodiments, in-vivo micro pump 100 using the Seldinger technique. In the Seldinger technique in-vivo micro pump 100 may be delivered to its implantation location in the aortic root by means of direct puncture of the heart apex 9 (FIG. 5A). An introducer sheath 11 having a dilator may be inserted into the left ventricle by means of apical puncture and advanced sheath 11 into the left ventricle (FIG. 5B). Introducer sheath 11 may further advance through the aortic valve and into position in the aorta (FIG. 5C). In-vivo micro pump 100 may then advance through sheath 11 causing expansion of an anchoring system 8 (FIGS. 5D and 5E). In some embodiments, following the anchoring of anchoring system 8, in-vivo micro pump 100 may be attached to a delivery wire 22, to be advanced in a catheter to its final location in its implantation location in the aortic root (FIG. 5F).

In some embodiments, in-vivo micro pump 100, once implanted and anchored in its appropriate position in a vessel is connected to the drive system by means of a cable inserted into the venous system by an appropriate Seldinger technique, or otherwise and advanced toward the right atrium by using a known guidewire-catheter technique. In the case of aortic implantation, illustrated in FIGS. 5A-5F, the drive cable is advanced by means of an atrial septal puncture technique with a subsequent septal closure device containing a tunnel for advancement of the drive cable into the left atrium, through the mitral valve into the left ventricle and connect by means of a magnetic attraction mechanism to the terminal of the drive cable coming from the impeller device.

In some embodiments, the drive and power box may be implanted using an approach similar to that of a pacemaker in an appropriate site under the skin such as the lower abdomen to be located close to the site of venous access. The drive box may contain any required electronic hardware and software with appropriate algorithms. The hardware and software may include a communication unit that may be configured to communicate with various sensors such as vascular pressure, heart rate, motion and the like. The sensory data may use, for example, to determine blood flow requirement and hence provide the adequate energy and other electrical parameter output to the micro-impeller pump to provide appropriate blood flow as required from time to time.

In some embodiments, the drive box may further include a rechargeable battery to provide backup energy to device's computer as well as to the impeller motor system, at least for short periods of time when the patient requires to be detached from the external power source. An induction charging system may be located on the surface of the box or detached and connected to the box but contained also underneath the skin permits transfer of electric power from outside the body to drive system without a physical connection through the skin.

In some embodiments, the communication unit may contain an antenna for communication by means of radiofrequency communication such as near-field communication, Bluetooth or alternatively by cellular communication or other long-distance communication method as determined to be appropriate including adequate encryption and security measures to prevent unauthorized access. As such, the entire device may be contained inside the body with no component traversing the skin reducing risk of complication such as infection and bleeding for example.

In some embodiments, the delivery of electric power to the device as mentioned above is provided by an induction lead attached on the skin over the corresponding induction loop receiver below the skin and affixed in location by, for example, magnetic attraction or a skin adhesive patch. This induction lead may directly receive power from a stationary power source, for example, when the patient is stationary or at rest. Alternatively, power may be provide by a set of wearable rechargeable batteries on a belt or similar wearable to provide energy when the patient is mobile or active.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. An in-vivo micro blood pump, comprising:
a holder, configured to be inserted into a blood vessel to be anchored to the blood vessel by one or more stents; and
two or more micro-impellers located inside the holder while having the same rotation axis,
wherein each micro-impeller comprises:
a rotor comprising:
a first hollow cylinder;
two or more internal blades, extending inward from a wall of the first hollow cylinder, wherein the radial dimension of each blade is less than an internal radius of the first hollow cylinder; and
one or more magnets; and
a cylindrical stator comprising:
one or more electromagnets,
wherein a first micro impeller has a first blades pitch, and a second micro impeller has a second blades pitch, different from the first blades pitch.

2. The in-vivo micro blood pump of claim 1, wherein the one or more magnets are embedded in the walls of the first hollow cylinder.

3. The in-vivo micro blood pump of claim 1, wherein the one or more electromagnets are embedded in a second hollow cylinder included in the cylindrical stator.

4. The in-vivo micro blood pump of claim 1, wherein when the electromagnets are provided with electrical power the rotor magnetically levitates.

5. The in-vivo micro blood pump of claim 1, wherein the holder is one of: a tube, a mesh and a stent.

6. The in-vivo micro blood pump of claim 1, further comprising a driveline configured to provide communication signals and electrical power to the two or more micro-impellers.

7. The in-vivo micro blood pump of claim 1, further comprising:
a controller configured to control the rotating speed of all the micro impellers.

8. The in-vivo micro blood pump of claim 1, further comprising:
one or more pressure sensors; and
a controller configured to control the rotating speed of at least one rotor based on a signal received from the one or more pressure sensors.
